(19) United States
(12) Patent Application Publication
(10) Pub. No.: US 2021/0241867 A1
(43) Pub. Date: Aug. 5, 2021

(54) SYSTEMS AND METHODS FOR ADVANCED TISSUE SCREENING

(71) Applicant: Mid-America Transplant Services d/b/a Mid-America Transplant, Saint Louis, MO (US)

(72) Inventors: Mark Harmon White, Chesterfield, MO (US); John Anthony Simpson, Florissant, MO (US)

(21) Appl. No.: 16/778,112

(22) Filed: Jan. 31, 2020

(51) Int. Cl. G16H 10/40; G06Q 10/10; G16H 10/60; G06F 16/22

(52) U.S. Cl. CPC G16H 10/40; G06F 16/22; G16H 10/60; G06Q 10/107

(57) ABSTRACT

A tissue screening (TS) computing device for managing tissue screening is provided. The TS computing device includes at least one processor in communication with at least one memory device. The at least one processor is programmed to (i) store tissue screening data associated with a plurality of tissue processors, the tissue screening data including a set of tissue screening parameters for each donor tissue request received by an organ procurement organization (OPO), (ii) receive, from the OPO, a search request to identify tissue processors that satisfy at least one search condition, (iii) compare the at least one search condition to each set of tissue screening parameters, (iv) generate search results in response to the received request, the search results including the at least one tissue processor, and (v) generate and transmit instructions for displaying a first graphical user interface including the search results to the user computing device.

### BACKGROUND

The field of the invention relates generally to a tissue screening system and, more particularly, to a network based real-time donor tissue matching and screening system and method for facilitating management of tissue screening parameters associated with requests for donor tissue and donor organs in real time.

Many organ procurement organizations (OPOs) receive high volumes of requests for donor tissues and donor organs from tissue processors requesting on behalf of transplant candidates who are waiting for a tissue match and/or an organ match. Tissue processors may provide screening parameters specifying various attributes of the donor as well as the tissue type or organ type needed. These parameters may change either regularly or based on changing needs. At least some tissue processors may provide additional updates or changes regarding one or more screening parameters. Some tissue processors may provide additional instructions regarding, for example, screening procedures and preferences. Accordingly, many OPOs are responsible for not only procuring tissues and organs from donors, but also for managing and tracking multiple different screening parameters associated with each tissue processor with which an OPO is associated with.

At least some known OPOs receive notifications of updates and changes regarding screening parameters, and/or new requests for donor tissue and donor organs by electronic mail (e.g., e-mail) or postal mail. Thus, a given OPO is responsible for manually tracking each update or change received from each tissue processor associated with the OPO. This manual process can be cumbersome and prone to error, which may delay or otherwise negatively affect tissue and organ placements with tissue processors. Accordingly, there exists a need for a real-time tissue screening system that enables OPOs to efficiently organize and accurately manage high volumes of donor tissue and donor organ requests.

### BRIEF DESCRIPTION

In one aspect, a tissue screening (TS) computing device for managing tissue screening is provided. The TS computing device includes at least one processor in communication with at least one memory device. The at least one processor is programmed to store, in a database associated with the TS computing device, tissue screening data associated with a plurality of tissue processors. The tissue screening data includes a set of tissue screening parameters for each donor tissue request received by an organ procurement organization (OPO) from the plurality of tissue processors. The at least one processor is also programmed to receive, from a user computing device associated with the OPO, a search request to identify one or more tissue processors that satisfy at least one search condition. The at least one processor is also programmed to compare the at least one search condition to each set of tissue screening parameters stored in the database to determine at least one tissue processor that matches the at least one search condition. The at least one processor is also programmed to generate, based on the comparison, search results in response to the received request. The search results include the at least one tissue processor. The at least one processor is further programmed to generate instructions for displaying a first graphical user interface on the user computing device. The first graphical user interface includes the search results. The at least one processor is further programmed to transmit the generated instructions to the user computing device to cause the first graphical user interface to be displayed on the user computing device.

In another aspect, a computer-implemented method for managing tissue screening is provided. The method is implemented using a tissue screening (TS) computing device. The TS computing device includes at least one processor in communication with at least one memory device. The method includes storing, in a database associated with the TS computing device, tissue screening data associated with a plurality of tissue processors. The tissue screening data includes a set of tissue screening parameters for each donor tissue request received by the OPO from the plurality of tissue processors. The method also includes receiving, by the TS computing device, from a user computing device associated with the OPO, a search request to identify one or more tissue processors that satisfy at least one search condition. The method also includes comparing, by the TS computing device, the at least one search condition to each set of tissue screening parameters stored in the database to determine at least one tissue processor that matches the at least one search condition. The method further includes generating, by the TS computing device, based on the comparison, search results in response to the received request. The search results include the at least one tissue processor. The method also includes generating instructions for displaying a first graphical user interface on the user computing device. The first graphical user interface includes the search results. The method also includes transmitting the generated instructions to the user computing device to cause the first graphical user interface to be displayed on the user computing device.

In yet another aspect, one or more non-transitory computer-readable storage media having computer-executable instructions embodied thereon is provided. When executed by at least one processor on a tissue screening (TS) computing device, the computer-executable instructions cause the at least one processor to store, in a database associated with the TS computing device, tissue screening data associated with a plurality of tissue processors. The tissue screening data includes a set of tissue screening parameters for each donor tissue request received by an OPO from the plurality of tissue processors. The computer-executable instructions also cause the at least one processor to receive, from a user computing device associated with the OPO, a search request to identify one or more tissue processors that satisfy at least one search condition. The computer-executable instructions also cause the at least one processor to compare the at least one search condition to each set of tissue screening parameters stored in the database to determine at least one tissue processor that matches the at least one search condition. The computer-executable instructions also cause the at least one processor to generate, based on the comparison, search results in response to the received request. The search results include the at least one tissue processor. The computer-executable instructions also cause the at least one processor to generate instructions for displaying a first graphical user interface on the user computing device. The first graphical user interface includes the search results. The computer-executable instructions also cause the at least one processor to transmit the generated instructions to the user computing device to cause the first graphical user interface to be displayed on the user computing device.

### BRIEF DESCRIPTION OF THE DRAWINGS

The Figures described below depict various aspects of the systems and methods disclosed therein. It should be understood that each Figure depicts an embodiment of a particular aspect of the disclosed systems and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and are instrumentalities shown, wherein:

FIG. 1 is an example screenshot of a first query results screen from a tissue screening (TS) computing device for an organ procurement organization (OPO) user interface in accordance with an example embodiment of the present disclosure.

FIG. 2 is an example screenshot of a second query results screen from the TS computing device for an OPO user interface in accordance with an example embodiment of the present disclosure.

FIG. 3 is an example screenshot of a first management screen from the TS computing device for an OPO user interface in accordance with an example embodiment of the present disclosure.

FIG. 4 is an example screenshot of a second management screen from the TS computing device for an OPO user interface in accordance with an example embodiment of the present disclosure.

FIG. 5 is an example screenshot of a third management screen from the TS computing device for an OPO user interface in accordance with an example embodiment of the present disclosure.

FIG. 6 is a simplified block diagram of an example tissue screening (TS) computer system used in tissue screening that includes a TS computing device, in accordance with one embodiment of the present disclosure.

FIG. 7 is an example configuration of a server system, such as the TS computing device of FIG. 6, in accordance with an example embodiment of the present disclosure.

FIG. 8 is an example configuration of a client computer device shown in FIG. 6, in accordance with an example embodiment of the present disclosure.

FIG. 9 is a flowchart of an example process for providing a tissue screening (TS) computer system of FIG. 6, in accordance with an example embodiment of the present disclosure.

### DETAILED DESCRIPTION OF THE DISCLOSURE

The systems and methods described herein are directed to a tissue screening (TS) system that includes a tissue screening (TS) server (e.g., a tissue screening computing device) for managing tissue screening in real time. In the example embodiment, a TS system includes a plurality of organ procurement organizations (OPOs) and a plurality of tissue processors (TPs).

Organ procurement organizations (OPOs) are organizations responsible for recovering and storing human organs and tissues from deceased donors for transplantation usually within the United States. At the time of writing, there are fifty-eight federally-mandated OPOs throughout the United States and its territories. Each OPO has arrangements in place with one or more tissue processors that process and place recovered human tissues and organs with transplant facilities (e.g., transplant center, hospital) which request donor tissues and/or donor organs for candidate recipients. Tissue processors may request specific types of organs and tissues from one or more OPOs. Tissue processors may contract with one or more OPOs to receive recovered tissue. A tissue processor provides, to OPOs, screening parameters regarding specific tissues and/or organs the tissue processor is interested in obtaining on behalf of, for example, a transplant candidate. When a tissue recovered by an OPO matches a particular tissue processor's screening parameters, the OPO sends the recovered tissue to the tissue processor. The tissue processor subsequently processes the recovered tissue into usable grafts, and places the recovered tissue with requesting transplant facilities for transplantation.

In the example embodiment, the TS computing device receives, from an OPO user associated with a particular OPO, screening parameters associated with tissue requests and organ requests (collectively referred to herein as donor requests) from tissue processors for transplantation. The TS computing device may also receive, from the OPO user, screening parameters for research-designated donor tissues and organs from research facilities (e.g., hospitals, universities) for research purposes. Screening parameters are stored in a database associated with the TS computing device. In some embodiments, a tissue processor may also register with the TS computing device to manage the OPOs associated with the tissue processor. In these embodiments, a registered tissue processor may provide screening parameters and any changes to previously-provided screening parameters directly to the TS computing device instead of, or in addition to, distributing the information to each OPO associated with the tissue processor.

Screening parameters associated with individual requests for donor tissue and donor organs from tissue processors may need to be edited (e.g., updated) to accommodate changes from tissue processors, as tissue processors obtain additional information from requesting transplant facilities regarding requested donor tissue or donor organs. Thus, an OPO is responsible for managing and updating multiple sets of parameters associated with donor requests (e.g., requests for donor tissue and donor organs) for each tissue processor associated with the OPO. Each donor request provided by a tissue processor to an OPO identifies the tissue or organ requested and further includes screening parameters associated with the requested tissue or organ. Screening parameters include donor specifications, such as age range, gender, and/or weight range of a donor. Screening parameters may further include specific screening instructions for a requested donor tissue or donor organ. In the example embodiment, tissue processors also provide an activation date (e.g., an effective start date), notifying OPOs when to start screening for the requested donor tissue or donor organ. Tissue processors may further provide a deactivation date (e.g., an effective end date), notifying OPOs when to stop screening for the requested donor tissue or donor organ.

In the example embodiment, when an OPO recovers or secures an opportunity to recover a donor tissue and/or donor organ for placement, the OPO utilize the TS computing device to identify tissue processors that may be interested in receiving the recovered tissue or organ. The TS computing device may identify interested tissue processors by comparing screening parameters associated with donor requests with search variables (e.g., search conditions) provided by the OPO for the recovered donor tissue or donor organ. The TS computing device is configured to rank the identified tissue processors with respect to one another based on priority preferences designated by a tissue processor (for multiple donor requests submitted by the same tissue processor) and based on priority preferences designated by the OPO regarding preferred tissue processors.

Additionally or alternatively, the OPO can utilize the TS computing device to search for donor requests based on search variables associated with, for example, a recovered donor tissue and/or donor organ. In the example embodiment, the TS computing device is configured to arrange and present search results by categories, such as organ and tissue, and sub-categories, such as organ type and tissue type, to allow the OPO to view and compare donor requests across categories (e.g., easily identify how many search results are for donor organs versus donor tissues) and within categories (e.g., easily identify that two search results for heart valves satisfy the search variables). The TS computing device may also rank donor requests that match a particular search condition with respect to one another based on priority preferences designated by tissue processors and/or based on priority preferences designated by the OPO for preferred tissue processors.

At least one of the technical solutions to the technical problems provided by this system may include: (i) improving accuracy and efficiency associated with managing donor requests and, more specifically, screening parameters associated with donor tissues and donor organs requested by tissue processors; (ii) facilitating communication between an OPO and the tissue processors in cooperation with the OPO; (iii) reducing errors associated with manually tracking donor requests and screening parameters; and/or (iv) improving user interaction with data associated with donor requests and tissue processors by organizing and presenting data into categories and sub-categories.

The methods and systems described herein may be implemented using computer programming or engineering techniques including computer software, firmware, hardware, or any combination or subset thereof, wherein the technical effects may be achieved by performing at least one of the following steps: (i) receiving, from a user computing device associated with an organ procurement organization (OPO), tissue screening data associated with a plurality of tissue processors, the screening data including a set of tissue screening parameters for each donor tissue request received by the OPO from the plurality of tissue processors; (ii) storing, in a database associated with the TS computing device, the received screening data, (iii) receiving, from the user computing device associated with the OPO, a search request to identify one or more tissue processors that satisfy at least one search condition; (iv) comparing the at least one search condition to each set of tissue screening parameters stored in the database to determine at least one tissue processor that matches the at least one search condition; (v) generating, based on the comparison, search results in response to the received request, the search results including the at least one tissue processor; (vi) generating instructions for displaying a first graphical user interface on the user computing device, the first graphical user interface including the search results; and/or (vii) transmitting the generated instructions to the user computing device to cause the first graphical user interface to be displayed on the user computing device.

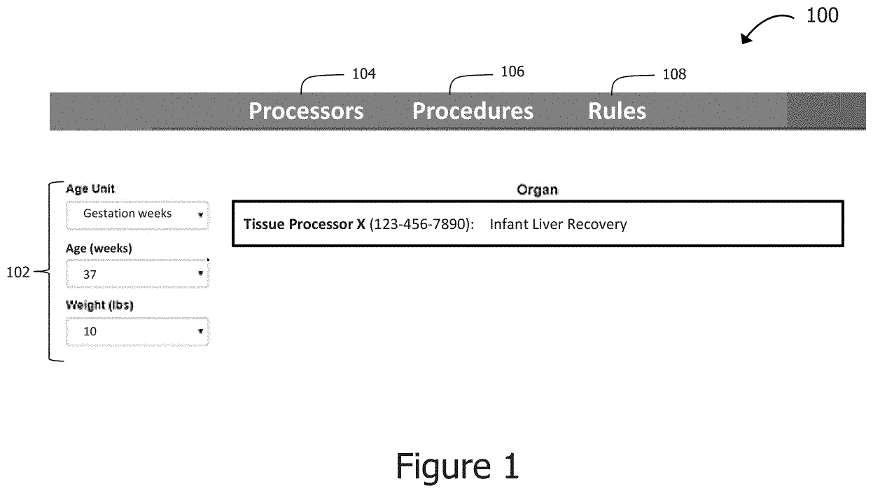

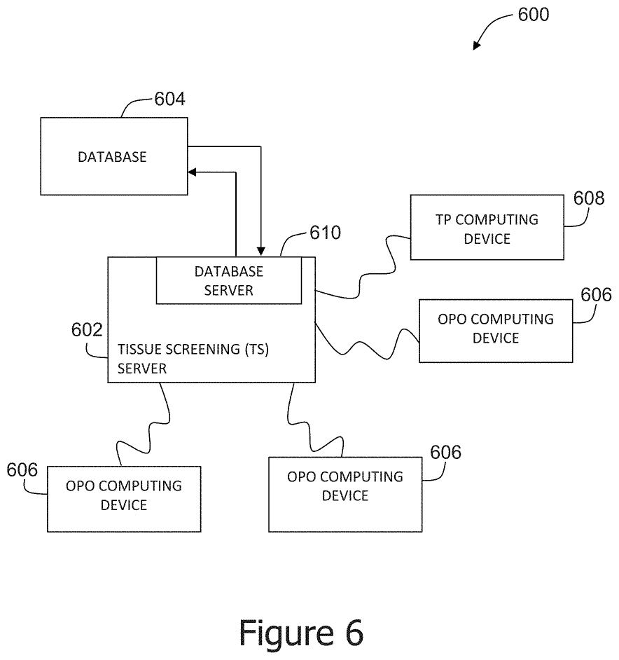

FIGS. 1-5 illustrate example screen captures or “screenshots” of an organ procurement organization (OPO) user interface in accordance with example embodiments of the present disclosure. In the example embodiment, an OPO registers with a tissue screening (TS) server 602 (shown in FIG. 6) to manage, at a central location, parameters of each tissue processor (TP) with which the OPO is in cooperation with. An OPO user associated with a registered OPO logs into TS system 600 (shown in FIG. 6) via a browser (not shown) installed on the OPO user's user computing device (e.g., OPO computing device 606, shown in FIG. 6) to access screens 100-500, as shown in FIGS. 1-5. An OPO user, as described herein, may refer to a tissue screener, organ screener, research screener, placement coordinator, procurement coordinator recovery coordinator, and/or any other OPO personnel authorized by an OPO (e.g., Mid-America Transplant Services, Louisiana Organ Procurement Agency) to access TS system 600 on behalf of the OPO.

FIG. 1 is an example screenshot of a first query result screen 100 from TS server 602 for an OPO user interface to be displayed on an OPO computing device (e.g., OPO computing device 606, shown in FIG. 6) in accordance with an example embodiment of the present disclosure. As shown in FIG. 1, TS server 602 allows the OPO user to search for interested tissue processors based on age unit (e.g., gestation weeks, days, years), age, and weight (e.g., grams, kilograms, pounds) via search fields 102. Search fields 102 may each include a drop-down menu of possible selections for the OPO user to choose from. In FIG. 1, the OPO user selects “gestation weeks” as the age unit, “37” weeks as the age, and “10” pounds (lbs.) as the weight. In response, TS server 602 queries its database (e.g., database 604, shown in FIG. 6) to identify tissue processors that (i) are associated with the OPO and (ii) satisfy the search variables provided by the OPO user.

Upon completion of the execution of the query, TS server 602 transmits instructions to OPO computing device 606 to display the results on first query result screen 100. As shown in FIG. 1, TS server 602 identifies a tissue processor interested in receiving a liver from an infant donor who has reached 37 gestations weeks and is 10 pounds. More specifically, first query result screen 100 provides the OPO user with a tissue processor identifier (“Tissue Processor X”), a contact number (“123-456-7890”) associated with tissue processor, and a description of the requested organ (“Infant Liver Recovery”) on first query result screen 100. The OPO user may call Tissue Processor X at the displayed contact number, and notify Tissue Processor X of the available donor organ (e.g., “We have a liver from a preborn donor who has reached 37 weeks of gestation and is 10 pounds—are you interested?”).

Notably, as shown on first query results screen 100, TS server 602 allows the OPO user to easily search for tissue processors interested in preborn tissue and organ donations by allowing the OPO user to search by gestations weeks as opposed to only days and/or years. Further, as donated organs, such as the liver, have a much shorter time frame than donated tissues to be recovered and transplanted (hours as opposed to days and/or years), TS server 602 allows OPO users to quickly identify and contact interested tissue processors for transplantation.

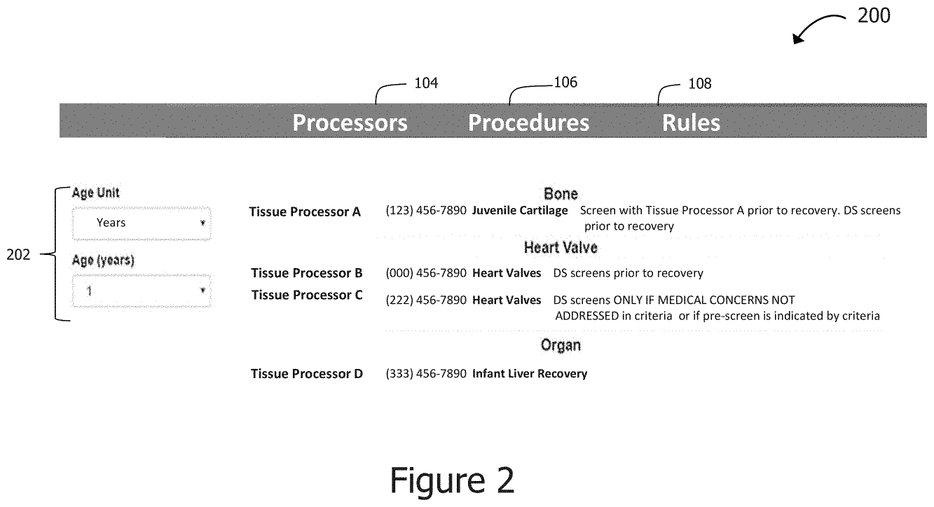

FIG. 2 is an example screenshot of a second query result screen 200 from TS server 602 for an OPO user interface to be displayed on an OPO computing device (e.g., OPO computing device 606, shown in FIG. 6) in accordance with an example embodiment of the present disclosure. In FIG. 2, the OPO user searches for tissue processors based on age (as opposed to both age and weight, as shown by search fields 102 in FIG. 1) via search fields 202. As shown in second query result screen 200, the OPO user selects “years” as the age unit and “1” year as the age to identify tissue processors interested in donated tissues or organs from donors who have reached one year of age. TS server 602 subsequently queries its database (e.g., database 604, shown in FIG. 6) to identify tissue processors that (i) are available to the OPO and (ii) satisfy the search variables provided by the OPO user. TS server 602 transmits instructions to OPO computing device 606 to display the search results on second query result screen 200 to allow the OPO user to view the results. In particular, second query result screen 200 displays four tissue processors that have submitted donor requests that match the search variables (“Tissue Processor A,” “Tissue Processor B,” “Tissue Processor C,” and “Tissue Processor D”). Similar to the search results displayed on first query result screen 100, the search results of second query result screen 200 also display a contact number for each tissue processor as well as a description of the requested organ or tissue requested.

TS server 602 is configured to retrieve additional screening instructions provided by a tissue processor that matches the search variables. TS server 602 transmits instructions to OPO computing device 606 to display the retrieved screening instructions as part of the search results on second query result screen 200. Second query result screen 200 displays specific screening instructions associated with Tissue Processor A, Tissue Processor B, and Tissue Processor C. For example, second query result screen 200 shows that Tissue Processor A has instructed the OPO associated with the OPO user to screen for juvenile cartilage prior to recovery. Similarly, second query result screen 200 shows that Tissue Processor B has instructed the OPO to screen for heart valves prior to recovery.

In FIG. 2, TS server 602 filters the search results into three categories (e.g., bone, heart valve, and organ) for display on the OPO user interface. Second query result screen 200 shows that two tissue processors (Tissue Processor B and Tissue Processor C) are both interested in heart valves from a donor who has reached 1 year of age. In one example, if the OPO user has recovered a limited number of heart valves from a donor who satisfies the screening parameters of both Tissue Processor B and Tissue Processor C, the OPO user is able to quickly assess that (i) two tissue processors are interested in the recovered heart valves and (ii) the OPO user needs to determine which tissue processor has priority of the recovered heart valves (e.g., which tissue processor the OPO user should call first).

With reference to FIGS. 1 and 2, in the example embodiment, TS server 602 transmits instructions to OPO computing device 606 to display a plurality of tabs, including a “processors” tab 104, a “procedures” tab 106, and a “rules” tab 108. “Processors” tab 104 enables the OPO user to view a list (not shown) of all the tissue processors available to the OPO associated with the OPO user. The user can select “processors” tab 104 to find a particular tissue processor. The user can select the tissue processor from the list to view tissue processor information (e.g., contact information, donor requests, and correspondences) associated with the selected tissue processor in the OPO user interface.

“Procedures” tab 106 enables the OPO user to view information associated with donor requests according to categories (e.g., procedure categories). The categories may include surgical procedures (e.g., dialysis, joint restoration), donor tissue (for donor tissue requests), donor organ (for donor organ requests), and research (for donor organs/tissues designated for research purposes). For example, donor requests for bone, tendons, cornea, veins, heart valve, and skin may be categorized under a “donor tissue request” category (not shown). In another example, donor requests for heart, kidneys, lungs, liver, pancreas, and intestines may be categorized under a “donor organ request” category (not shown). Within each category, donor request information may be further arranged into sub-categories (e.g., procedures). Additionally, within each sub-category, donor request information may be further arranged into sub-sub-categories. In one embodiment, donor tissue requests may be categorized into sub-categories, such as musculoskeletal tissue (e.g., bone, cartilage), dermal (e.g., full thickness, split thickness), soft tissue (e.g., tendons, ligaments, nerves), cardiovascular tissue (e.g., heart valves, veins), and ocular tissue (e.g., cornea, sclera). Sub-categories and sub-sub categories may be referred to herein as procedures. In one example, sub-categories including traditional, osteocel, juvenile cartilage, joint restoration, and adipose may be categorized under a “bones” category.

TS server 602 may arrange information associated with donor requests from multiple tissue processors into approachable categories and sub-categories to allow the OPO user to efficiently navigate and manage donor requests received from tissue processors. In embodiments where recovered donor organs and/or donor tissues are not suitable for transplantation, but are suitable for research, the recovered donor organs and/or donor tissues may be grouped under the research category. In these embodiments, an OPO user, such as a research screener, may select the research category from “procedures” tab 106 to view all the donor organs and tissues designated for research.

Within the research category, organs and tissues may further be organized under sub-categories based on, for example, serology data (e.g., whether the recovered organ or tissue is diseased or not diseased). Diseased organs and tissues may further be categorized by type of disease or infection (e.g., Hepatitis C). Accordingly, in these embodiments, OPO users can place organs and/or tissues designated for research based on research parameters provided by a requesting researching facility (e.g., universities, hospitals). Research parameters may include a disease type and an organ or tissue type requested by a researching facility (e.g., “We are conducting research on Hepatitis C infections in kidneys and are interested in obtaining donor a kidney infected with Hepatitis C”). For example, if an OPO recovers or secures an opportunity to recover a Hepatitis C-infected kidney, the OPO may access the research tab from “procedures” tab 106 to identify researching facilities interested in receiving the infected kidney.

In the example embodiment, “rules” tab 108 enables the OPO user to access screening parameters associated with donor requests from tissue processors associated with a given OPO. The OPO user associated with the OPO may update a particular tissue processor's screening parameters for donor requests associated with the tissue processor by accessing “rules” tab 108. In other embodiments, the OPO user may update a tissue processor's screening parameters for a particular donor request by accessing “procedures” tab 106.

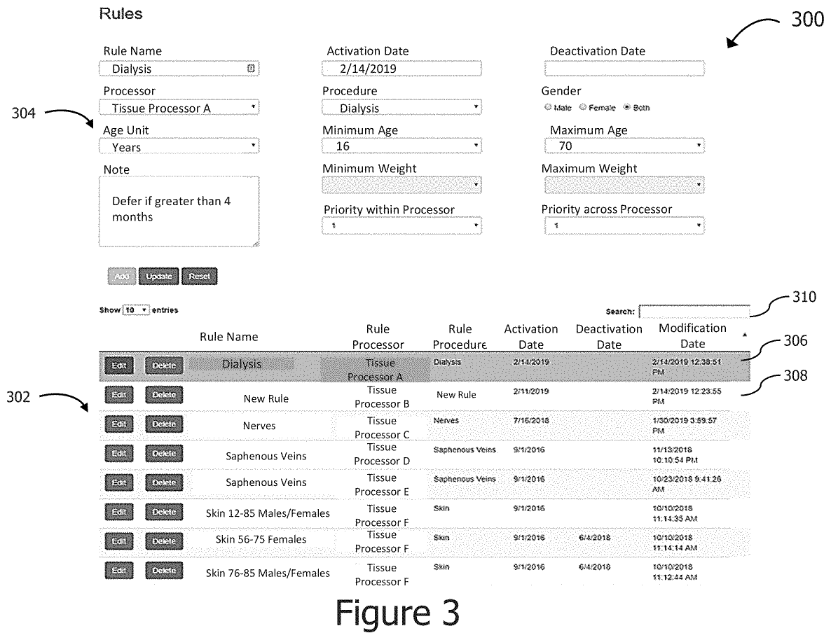

In the example embodiment, the OPO user is able to manage screening parameters associated with donor requests provided by tissue processors, as shown in FIGS. 3-5. FIGS. 3-5 are example screenshots of management screens from TS server 602 for an OPO user interface to be displayed on an OPO computing device (e.g., OPO computing device 606, shown in FIG. 6) in accordance with an example embodiment of the present disclosure. More specifically, FIG. 3 depicts a rules management screen 300 including a rules section 302 and a rule update section 304. More specifically, the OPO user may access rules management screen 300 by selecting “rules” tab 108 (shown in FIGS. 1 and 2) in the OPO user interface. Rules section 302 provides a list of rule entries that correspond to donor requests received by the OPO.

As shown in rules management screen 300, rules section 302 provides rows of rule entries associated with donor requests. Each rule entry corresponds to a set of screening parameters associated with a donor request received by an OPO from a particular tissue processor. Rules section 302 displays, for each rule entry, an entry name, a tissue processor (e.g., a rule processor), a category (e.g., a rule procedure), an activation date, a deactivation date (if applicable), and an entry modification date.

An “edit” option and a “delete” option are provided next to each rule entry displayed in rules section 302. The OPO user can delete or edit one or more rule entries displayed in rules section 302. In the example embodiment, when the OPO user selects the “edit” option for a specific rule entry, TS server 602 transmits instructions to OPO computing device 606 to populate the parameter fields of rule update section 304 with stored parameters for the selected rule entry. In FIG. 3, a first rule entry 306 is selected from rules section 302 for editing in rule update section 304. The OPO user can proceed to edit one or more fields displayed in rule update section 304 for first rule entry 306. More specifically, in rule update section 304, the OPO user may add a deactivation date, change the minimum and/or maximum age, add a minimum and/or maximum weight, add additional notes, edit the entry name, and/or edit the priority preference for first rule entry 306.

Rule update section 304 provides a “priority within processor” parameter field and a “priority across processor” parameter field to allow the OPO user to designate priority preferences both within a tissue processor and across tissue processors associated with the OPO. Both parameter fields provide a drop-down menu of selection choices to allow the OPO user to select a priority preference in numerical form (e.g., first priority is designated as “1” and second priority is designated as “2”). The OPO user designates a priority preference for the “priority within processor” parameter field in accordance with priority instructions received from a tissue processor regarding multiple donor tissues and/or organs.

In one example, a tissue processor identifies three donor tissues (e.g., cartilage, heart valves, and saphenous veins) the tissue processor is interested in obtaining from donors between the ages of 2 and 18. In this example, the tissue processor may instruct the OPO to notify the tissue processor of available donor cartilage before the OPO notifies the tissue processor of available donor heart valves or saphenous veins. The tissue processor may further instruct the OPO to notify the tissue processor of available donor heart valves before the OPO notifies the tissue processor of available donor saphenous veins. Accordingly, the priority preference for the “priority within processor” parameter field is designated according to priority preferences established by the tissue processor (e.g., “Call us about available donor cartilages first, available donor heart valves second, and available donor saphenous veins last”). The “priority within processor” parameter field is used to establish a single tissue processor's priority preference within a particular category of donor tissue and/or donor organ.

In contrast, the “priority across processor” field is designated according to priority preferences established by the OPO regarding which tissue processor to call first when an available donor tissue or donor organ satisfies the screening parameters of multiple tissue processors. Thus, the “priority across processor” parameter field reflects the OPO's priority preference among tissue processors. In particular, the “priority across processor” parameter field is used to establish the OPO's priority preference with respect to tissue processors for a particular category of donor tissue and/or donor organ. The OPO may have closer working relationships with certain tissue processors over others. Priority preferences across tissue processors may be based on geographic location (e.g., local, regional, national). For example, an OPO's priority preference may be set such that a tissue processor located twenty miles away is given an opportunity to receive a requested donor tissue or donor organ before the OPO contacts another tissue processor located two hundred miles away. Priority preferences may also be based on factors such as, whether a given tissue processor is not-for-profit or for-profit, and whether a given tissue processor is an OPO, as some OPOs also operate as tissue processors. In some embodiments, priority preference may be based on demand or need for a particular donor tissue and/or donor organ. For example, a tissue processor may determine that they have too many donor tissues that meet a specific criteria. In this example, the tissue processor may specifically instruct the OPO to designate the tissue processor as a low priority for this particular type of donor tissue for a designated period of time.

When an OPO receives a new donor request from a tissue processor, an OPO user may create a new rule entry for the search parameters provided in the new donor request in rule update section 304. New rule entries appear in rules section 302, as shown by second entry 308 on rules section 302. In the example embodiment, rules management screen 300 also includes a search function, such as search function 310, which enables an OPO user to search for a particular set of search parameters associated with a donor request.

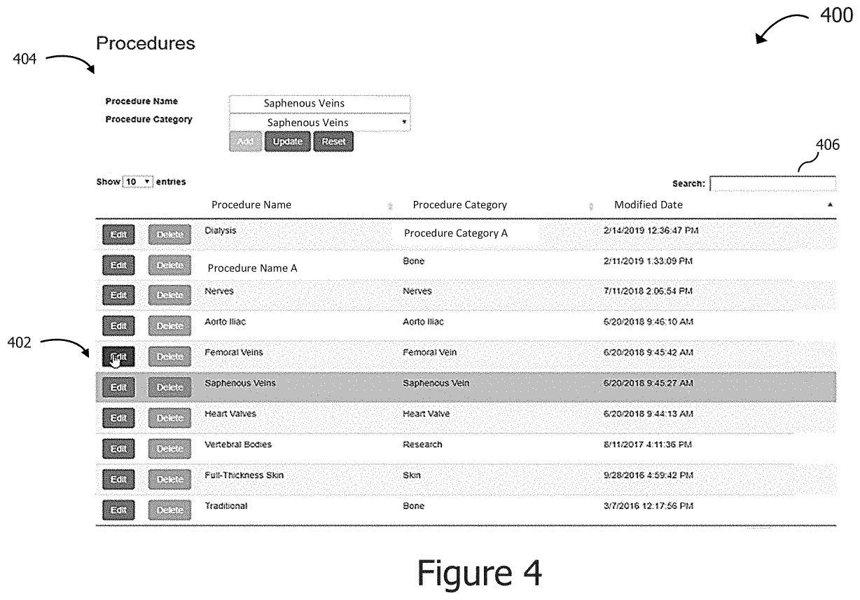

FIG. 4 depicts a sub-categories management screen 400 including a procedures section 402 and a procedure update section 404. Procedures section 402 provides a list of entries associated with sub-categories (e.g., procedures) designated for a given tissue processor. In particular, procedures section 402 displays a list of sub-categories used to organize donor request information from donor requests associated with a tissue processor. Procedures section 402 displays, for each entry, an entry name (e.g., an entry identifier), a category associated with the sub-category, and an entry modification date. The OPO user can edit or remove a sub-category in procedures section 402 by selecting one of an “edit” option or a “delete” option provided next to each entry. In FIG. 4, an entry for a sub-category labeled “Saphenous Veins” is selected for editing in procedure update section 404. Procedure update section 404 shows the broader category (e.g., procedure category) in which the selected sub-category is associated with.

An OPO user may create a new sub-category for a given tissue processor in procedure update section 404. When creating a new procedure entry, the OPO user selects a broader category (e.g., procedure category) for which the new sub-category is to be associated with. In the example embodiment, sub-categories management screen 400 also includes a search function, such as search function 406, which enables an OPO user to search for a particular sub-category associated with a given tissue processor.

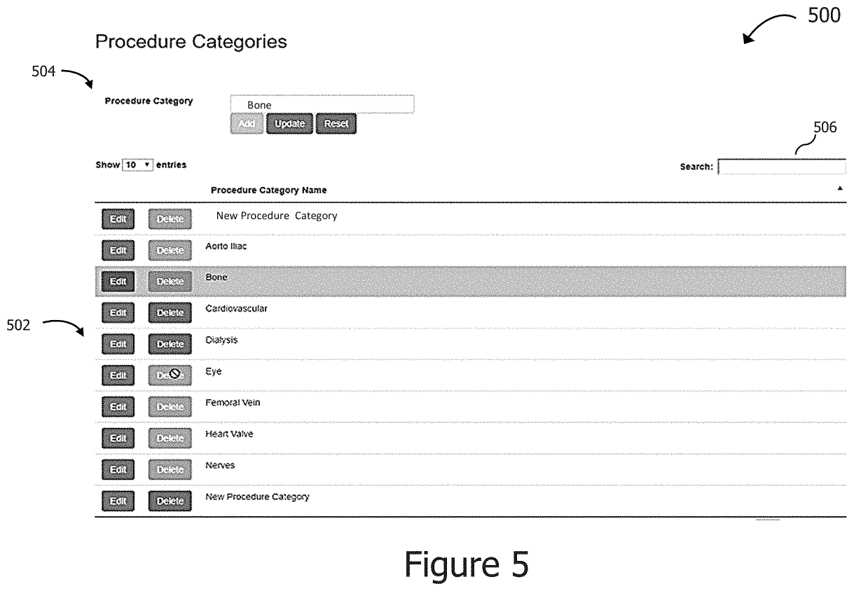

FIG. 5 depicts a categories management screen 500 including a categories section 502 and a category update section 504. Categories section 502 provides a list of entries associated with broader categories (e.g., procedure categories) for a given tissue processor. Categories section 502 allows an OPO user to edit or remove a category by providing an “edit” option and a “delete” option next to each category entry. In FIG. 5, a category entry labeled “Bone” is selected for editing in category update section 504. Category update section 504 allows the OPO user to change a selected category entry's name. Category update section 504 allows the OPO user to create new categories, as shown by the entries labeled “New Procedure Category” in categories section 502. In the example embodiment, categories management screen 500 also includes a search function, such as search function 506, which enables an OPO user to search for a particular category associated with a given tissue processor.

In embodiments where tissue processors register with TS server 602, TS server 602 may generate instructions for displaying a tissue processor (TP) user interface (not shown) on a tissue processor's user computing device, such as TP computing device 608 (shown in FIG. 6). The TP user interface may include a list of OPOs associated with a registered tissue processor. The TP user interface may further include donor requests, screening parameters, and/or priority preferences provided by the tissue processor to each associated OPO. TS server 602 may allow the tissue processor to update one or more screening parameters provided to an OPO.

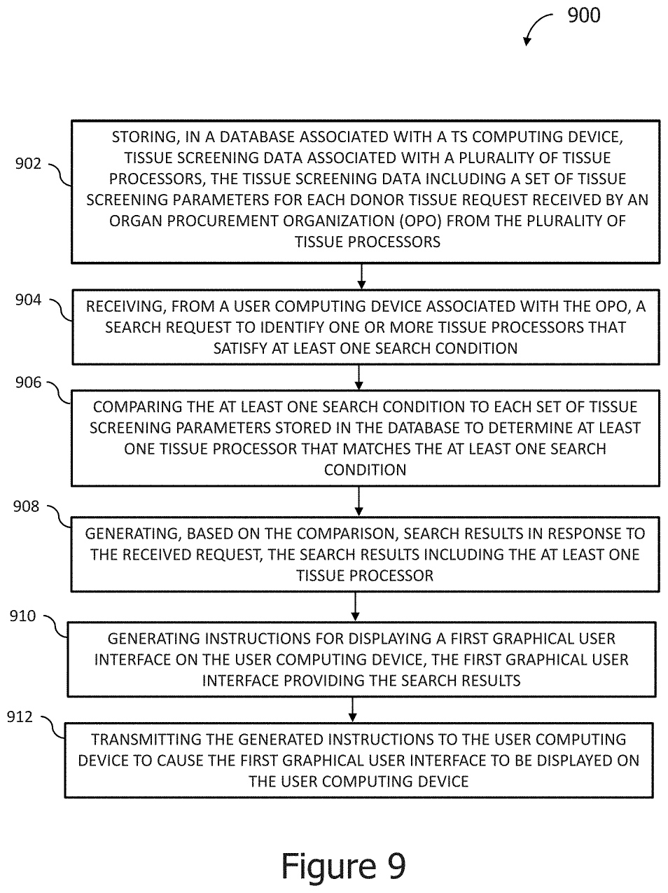

FIG. 6 depicts a simplified block diagram of an example tissue screening (TS) system 600 for implementing method 900 shown in FIG. 9. In the example embodiment, system 600 may be used to manage donor requests (e.g., donor tissue requests, donor organ requests), and more specifically, to manage the screening parameters associated with each donor request received by an organ procurement organization (OPO) from tissue processors. System 600 includes a tissue screening (TS) server (e.g., a tissue screening (TS) computing device) 602, and may be used to manage requests for research-designated donor tissues and donor organs from research facilities. As described herein, TS server 602 may be configured to (i) store, in a database, tissue screening data associated with a plurality of tissue processors, the tissue screening data including a set of tissue screening parameters for each donor tissue request received by an OPO from the plurality of tissue processors; (ii) receive, from a user computing device associated with the OPO, a search request to identify one or more tissue processors that satisfy at least one search condition; (iii) compare the at least one search condition to each set of tissue screening parameters stored in the database to determine at least one tissue processor that matches the at least one search condition; (iv) generate, based on the comparison, search results in response to the received request, the search results including the at least one tissue processor; (v) generate instructions for displaying a first graphical user interface on the user computing device, the first graphical user interface including the search results; and/or (vi) transmit the generated instructions to the user computing device to cause the first graphical user interface to be displayed on the user computing device.

In the example embodiment, organ procurement organization (OPO) computing devices 606 and tissue processor (TP) computing devices 608 are computers that include a web browser or a software application, which enables OPO computing devices 606 and TP computing devices 608 to access remote computer devices, such as TS server 602 using the Internet or other network. More specifically, OPO computing devices 606 and TP computing devices 608 may be communicatively coupled to the Internet through many interfaces including, but not limited to, at least one of a network, such as the Internet, a local area network (LAN), a wide area network (WAN), or an integrated services digital network (ISDN), a dial-up-connection, a digital subscriber line (DSL), a cellular phone connection, and a cable modem. OPO computing devices 606 and TP computing devices 608 may be any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices.

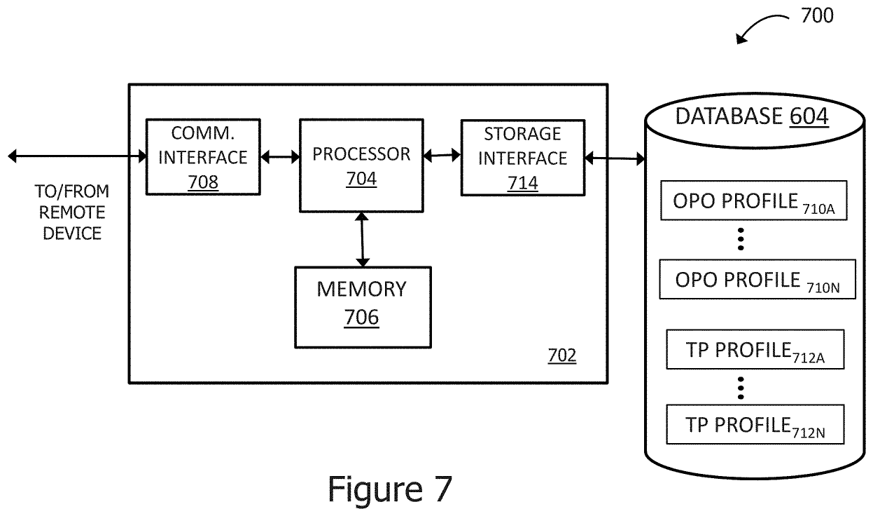

A database server 610 is communicatively coupled to a database 604 that stores data. In one embodiment, database 604 may include OPO profiles associated with registered OPOs (e.g., OPO profiles 710, shown in FIG. 7). An OPO profile for a given OPO may include a list of tissue processors associated with the OPO, donor requests received from each tissue processor, screening parameters (e.g., donor age, donor gender, donor weight) associated with each donor request, an activation date and a deactivation date associated with each set of screening parameters, priority preferences within each tissue processor regarding requested donor tissues and/or donor organs, and priority preferences across the tissue processors associated with the OPO for tissues and/or organs. In another embodiment, database 604 may include TP profiles associated with registered tissue processors (e.g., TP profiles 712, shown in FIG. 7). A TP profile for a given tissue processor may include a list of OPOs associated with the tissue processor, donor requests sent to each OPO, communications sent to each OPO regarding any changes or updates to sets of screening parameters, and additional instructions and/or notes regarding each donor request.

In some embodiments, database 604 is stored remotely from TS server 602. In some embodiments, database 604 is decentralized. In some embodiments, database 604 includes any computer server, cloud or other digital data storage device. In the example embodiment, an OPO user may access database 604 via OPO computing device 606 by logging onto TS server 602, as described herein. In embodiments where tissue processors register with TS server 602, tissue processors may access database 604 via TP computing devices 608 by logging onto TS server 602.

TS server 602 may be in communication with a plurality of OPO computing devices 606 and a plurality of TP computing devices 608 to manage donor tissue and donor organ information received from OPO computing devices 606 and TP computing devices 608 in real time. In the example embodiment, TS server 602 is a computer that allows remote computers that include a web browser or a software application, such as OPO computing devices 606 and TP computing devices 608, to access for communication, using the Internet or other network. In some embodiments, TS server 602 is a remote computing device accessed in the execution of an application installed on OPO computing device 606 and/or TP computing device 608. More specifically, TS server 602 may be communicatively coupled to the Internet through many interfaces including, but not limited to, at least one of a network, such as the Internet, a local area network (LAN), a wide area network (WAN), or an integrated services digital network (ISDN), a dial-up-connection, a digital subscriber line (DSL), a cellular phone connection, and a cable modem.

FIG. 7 illustrates an example configuration 700 of a server computing device 702, such as TS server 602, in accordance with one embodiment of the present disclosure. Server computer device 702 includes a processor 704 for executing instructions. Instructions may be stored in a memory area 706, for example. Processor 704 may include one or more processing units (e.g., in a multi-core configuration) configured to perform method 900 shown in FIG. 9.

Processor 704 may be operatively coupled to a communication interface 708 such that server computer device 702 is capable of communicating with a remote device such as one or more OPO computing devices 606 and TP computing devices 608 (both shown in FIG. 6) (for example, using wireless communication or data transmission over one or more radio links or digital communication channels). For example, communication interface 708 may receive, from OPO computing devices 606, via the Internet, sets of screening parameters associated with donor requests, changes to existing screening parameters, and search variables associated with query requests, as illustrated in FIGS. 1-5.

Processor 704 is also operatively coupled to a storage device, such as database 604. Database 604 may be any computer-operated hardware suitable for storing and/or retrieving data. With reference to FIG. 6, database 604 may include a plurality of OPO profiles 710A-710N (collectively, “OPO profiles 710”) associated with organ procurement organizations (OPOs) as well as a plurality of TP profiles 712A-712N (collectively, “TP profiles 712”) associated with tissue processors (TPs) registered with TS server 602 (shown in FIG. 6). TS server 602 periodically updates OPO profiles 710 and TP profiles 712 to include the most current changes to screening parameters associated with donor requests from tissue processors.

In some embodiments, database 604 may be integrated in server computer device 702. For example, server computer device 702 may include one or more hard disk drives as a storage device. In other embodiments, database 604 may be external to server computer device 702 and may be accessed by a plurality of server computer devices 702. For example, database 604 may include a storage area network (SAN), a network attached storage (NAS) system, and/or multiple storage units such as hard disks and/or solid state disks in a redundant array of inexpensive disks (RAID) configuration.

In some embodiments, processor 704 is operatively coupled to database 604 via a storage interface 714. Storage interface 714 is any component capable of providing processor 704 with access to database 604. Storage interface 714 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 704 with access to database 604.

Memory area 706 may include, but is not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are for example only, and are thus not limiting as to the types of memory usable for storage of a computer program.

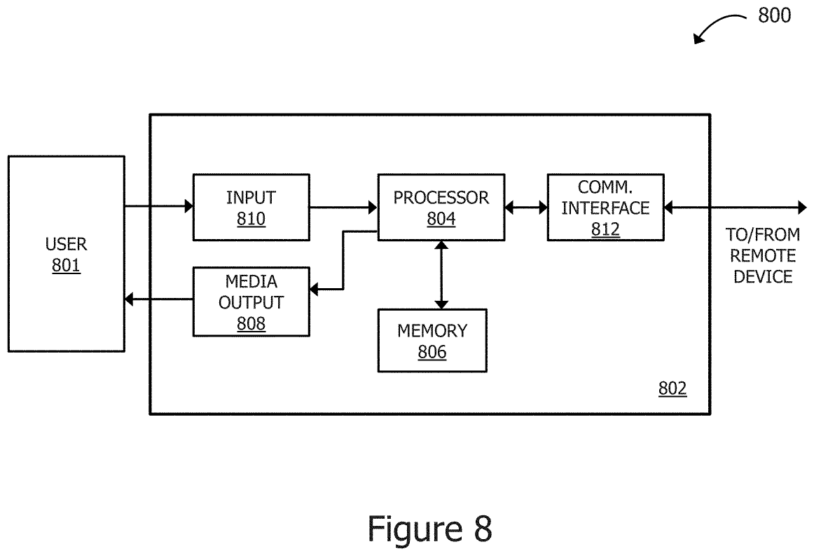

FIG. 8 depicts an example configuration of a remote or user computer device 802, such as OPO computing device 606s and TP computing devices 608 (both shown in FIG. 6), in accordance with one embodiment of the present disclosure. User computer device 802 may be operated by a user 801. User computer device 802 may include a processor 804 for executing instructions. In some embodiments, executable instructions may be stored in a memory area 806. Processor 804 may include one or more processing units (e.g., in a multi-core configuration). Memory area 806 may be any device allowing information such as executable instructions and/or transaction data to be stored and retrieved. Memory area 806 may include one or more computer readable media.

User computer device 802 may also include at least one media output component 808 for presenting information to user 801. Media output component 808 may be any component capable of conveying information to user 801. In some embodiments, media output component 808 may include an output adapter (not shown) such as a video adapter and/or an audio adapter. An output adapter may be operatively coupled to processor 804 and operatively coupleable to an output device such as a display device (e.g., a cathode ray tube (CRT), liquid crystal display (LCD), light emitting diode (LED) display, or “electronic ink” display) or an audio output device (e.g., a speaker or headphones).

In some embodiments, media output component 808 may be configured to present a graphical user interface (e.g., a web browser and/or a client application) to user 801. A graphical user interface may include, for example, an organ procurement organization (OPO) user interface for accessing a tissue screening application. In some embodiments, user computer device 802 may include an input device 810 for receiving input from user 801. User 801 may use input device 810 to, without limitation, provide and/or access sets of screening parameters associated with donor requests from tissue processors associated with an OPO.

Input device 810 may include, for example, a keyboard, a pointing device, a mouse, a stylus, a touch sensitive panel (e.g., a touch pad or a touch screen), a gyroscope, an accelerometer, a position detector, a biometric input device, and/or an audio input device. A single component such as a touch screen may function as both an output device of media output component 808 and input device 810.

User computer device 802 may also include a communication interface 812, communicatively coupled to a remote device such as TS server 602 (shown in FIG. 6). Communication interface 812 may include, for example, a wired or wireless network adapter and/or a wireless data transceiver for use with a mobile telecommunications network.

Stored in memory area 806 are, for example, computer readable instructions for providing a user interface to user 801 via media output component 808 and, optionally, receiving and processing input from input device 810. A user interface may include, among other possibilities, a web browser and/or a client application. Web browsers enable users, such as user 801, to display and interact with media and other information typically embedded on a web page or a website from TS server 602. A client application may allow user 801 to interact with, for example, TS server 602. For example, instructions may be stored by a cloud service, and the output of the execution of the instructions sent to the media output component 808.

FIG. 9 is a flowchart of an example method 900 for providing a tissue screening (TS) computing system, such as system 600 (shown in FIG. 6). In the example embodiment, method 900 is performed by a tissue screening (TS) computing device, such as TS server 602 (shown in FIG. 6). In certain embodiments, method 900 may be at least partially performed by a different computing device. In other embodiments, method 900 may include additional, fewer, or alternative actions, including those described elsewhere herein.

Method 900 begins with storing 902 tissue screening data associated with a plurality of tissue processors in a database, such as database 04 (shown in FIG. 6). The tissue screening data includes a set of tissue screening parameters for each donor tissue request received by an organ procurement organization (OPO) from the plurality of tissue processors. Method 900 also includes receiving 904, from a user computing device associated with the OPO, a search request to identify one or more tissue processors that satisfy at least one search condition. Method also includes comparing 906 the at least one search condition to each set of tissue screening parameters stored in the database to determine at least one tissue processor that matches the at least one search condition.

Method 900 also includes generating 908, based on the comparison, search results in response to the received request. The search results include the at least one tissue processor. Method 900 also includes generating 910 instructions for displaying a first graphical user interface on the user computing device. The first graphical user interface includes the search results. Method 900 further includes transmitting 912 the generated instructions to the user computing device to cause the first graphical user interface to be displayed on the user computing device. In some embodiments, the first graphical user interface includes a contact number associated with the at least one tissue processor and a tissue type of the donor tissue requested by the at least one tissue processor. In other embodiments, the at least one search condition includes a donor age provided in units of gestation weeks.

In some embodiments, method 900 also includes, prior to receiving the search request, (i) generating instructions for displaying a graphical interface widget on the user computing device, the graphical interface widget configured for inputting the at least one search condition in units of gestation weeks and/or (ii) transmitting the generated instructions to the user computing device to cause the graphical interface widget to be displayed on the user computing device. In some embodiments, method 900 also includes determining that a subset of the plurality of tissue processors match the at least one search condition and/or ranking the tissue processors within the subset with respect to one another based on priority rules designated by the OPO.

In some embodiments, method 900 includes determining that a subset of the plurality of donor tissue requests stored in the database match the at least one search condition and/or determining that the subset of donor tissue requests are associated with one tissue processor. In some embodiments, method 900 includes ranking the donor tissue requests within the subset with respect to one another based on priority rules established by the one tissue processor.

As will be appreciated based upon the foregoing specification, the above-described embodiments of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are example only, and are thus not intended to limit in any way the definition and/or meaning of the term “processor.”

As used herein, the term “database” may refer to either a body of data, a relational database management system (RDBMS), or to both. As used herein, a database may include any collection of data including hierarchical databases, relational databases, flat file databases, object-relational databases, object-oriented databases, and any other structured or unstructured collection of records or data that is stored in a computer system. The above examples are not intended to limit in any way the definition and/or meaning of the term database. Examples of RDBMS's include, but are not limited to, Oracle® Database, MySQL, IBM® DB2, Microsoft® SQL Server, Sybase®, and PostgreSQL. However, any database may be used that enables the systems and methods described herein. (Oracle is a registered trademark of Oracle Corporation, Redwood Shores, Calif.; IBM is a registered trademark of International Business Machines Corporation, Armonk, N.Y.; Microsoft is a registered trademark of Microsoft Corporation, Redmond, Wash.; and Sybase is a registered trademark of Sybase, Dublin, Calif.)

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

In another embodiment, a computer program is provided, and the program is embodied on a computer-readable medium. In an example embodiment, the system is executed on a single computer system, without requiring a connection to a server computer. In a further example embodiment, the system is being run in a Windows® environment (Windows is a registered trademark of Microsoft Corporation, Redmond, Wash.). In yet another embodiment, the system is run on a mainframe environment and a UNIX® server environment (UNIX is a registered trademark of X/Open Company Limited located in Reading, Berkshire, United Kingdom). In a further embodiment, the system is run on an iOS® environment (iOS is a registered trademark of Cisco Systems, Inc. located in San Jose, Calif.). In yet a further embodiment, the system is run on a Mac OS® environment (Mac OS is a registered trademark of Apple Inc. located in Cupertino, Calif.). In still yet a further embodiment, the system is run on Android® OS (Android is a registered trademark of Google, Inc. of Mountain View, Calif.). In another embodiment, the system is run on Linux® OS (Linux is a registered trademark of Linus Torvalds of Boston, Mass.). The application is flexible and designed to run in various different environments without compromising any major functionality.

In some embodiments, the system includes multiple components distributed among a plurality of computer devices. One or more components may be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes. The present embodiments may enhance the functionality and functioning of computers and/or computer systems.

As used herein, an element or step recited in the singular and preceded by the word “a” or “an” should be understood as not excluding plural elements or steps, unless such exclusion is explicitly recited. Furthermore, references to “example embodiment,” “example embodiment,” or “one embodiment” of the present disclosure are not intended to be interpreted as excluding the existence of additional embodiments that also incorporate the recited features.

Furthermore, as used herein, the term “real-time” refers to at least one of the time of occurrence of the associated events, the time of measurement and collection of predetermined data, the time to process the data, and the time of a system response to the events and the environment. In the embodiments described herein, these activities and events occur substantially instantaneously.

The patent claims at the end of this document are not intended to be construed under 35 U.S.C. § 112(f) unless traditional means-plus-function language is expressly recited, such as “means for” or “step for” language being expressly recited in the claim(s).

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A tissue screening (TS) computing device including at least one processor in communication with at least one memory device, the at least one processor programmed to:
store, in a database associated with the TS computing device, tissue screening data associated with a plurality of tissue processors, the tissue screening data including a set of tissue screening parameters for each donor tissue request received by an organ procurement organization (OPO) from the plurality of tissue processors;
receive, from a user computing device associated with the OPO, a search request to identify one or more tissue processors that satisfy at least one search condition;
compare the at least one search condition to each set of tissue screening parameters stored in the database to determine at least one tissue processor that matches the at least one search condition;
generate, based on the comparison, search results in response to the received request, the search results including the at least one tissue processor;
generate instructions for displaying a first graphical user interface on the user computing device, the first graphical user interface including the search results; and
transmit the generated instructions to the user computing device to cause the first graphical user interface to be displayed on the user computing device.

2. The TS computing device of claim 1, wherein the first graphical user interface includes a contact number associated with the at least one tissue processor and a tissue type of the donor tissue requested by the at least one tissue processor.

3. The TS computing device of claim 1, wherein the at least one search condition includes a donor age provided in units of gestation weeks.

4. The TS computing device of claim 1, wherein prior to receiving the search request, the at least one processor is further programmed to:
generate instructions for displaying a graphical interface widget on the user computing device, the graphical interface widget configured for inputting the at least one search condition in units of gestation weeks; and
transmit the generated instructions to the user computing device to cause the graphical interface widget to be displayed on the user computing device.

5. The TS computing device of claim 1, wherein the at least one processor is further programmed to:
determine that a subset of the plurality of tissue processors match the at least one search condition; and
rank the tissue processors within the subset with respect to one another based on priority rules designated by the OPO.

6. The TS computing device of claim 1, wherein the at least one processor is further programmed to:
determine that a subset of the plurality of donor tissue requests stored in the database match the at least one search condition; and
determine that the subset of donor tissue requests are associated with one tissue processor.

7. The TS computing device of claim 6, wherein the at least one processor is further programmed to rank the donor tissue requests within the subset with respect to one another based on priority rules established by the one tissue processor.

8. A computer-implemented method for managing tissue screening, the method implemented using a tissue screening (TS) computing device, wherein the TS computing device comprises at least one processor in communication with at least one memory device, the method comprising:
storing, in a database associated with the TS computing device, tissue screening data associated with a plurality of tissue processors, the tissue screening data including a set of tissue screening parameters for each donor tissue request received by an organ procurement organization (OPO) from the plurality of tissue processors;
receiving, by the TS computing device, from a user computing device associated with the OPO, a search request to identify one or more tissue processors that satisfy at least one search condition;
comparing, by the TS computing device, the at least one search condition to each set of tissue screening parameters stored in the database to determine at least one tissue processor that matches the at least one search condition;
generating, by the TS computing device, based on the comparison, search results in response to the received request, the search results including the at least one tissue processor;
generating instructions for displaying a first graphical user interface on the user computing device, the first graphical user interface including the search results; and
transmitting the generated instructions to the user computing device to cause the first graphical user interface to be displayed on the user computing device.

9. The computer-implemented method of claim 8, wherein the first graphical user interface includes a contact number associated with the at least one tissue processor and a tissue type of the donor tissue requested by the at least one tissue processor.

10. The computer-implemented method of claim 8, wherein the at least one search condition includes a donor age provided in units of gestation weeks.

11. The computer-implemented method of claim 8, wherein prior to receiving the search request, the method further comprises:
generating, by the TS computing device, instructions for displaying a graphical interface widget on the user computing device, the graphical interface widget configured for inputting the at least one search condition in units of gestation weeks; and
transmitting the generated instructions to the user computing device to cause the graphical interface widget to be displayed on the user computing device.

12. The computer-implemented method of claim 8 further comprising:
determining, by the TS computing device, that a subset of the plurality of tissue processors match the at least one search condition; and
ranking, by the TS computing device, the tissue processors within the subset with respect to one another based on priority rules designated by the OPO.

13. The computer-implemented method of claim 8 further comprising:
determining, by the TS computing device, that a subset of the plurality of donor tissue requests stored in the database match the at least one search condition; and
determining, by the TS computing device, that the subset of donor tissue requests are associated with one tissue processor.

14. The computer-implemented method of claim 13 further comprising ranking, by the TS computing device, the donor tissue requests within the subset with respect to one another based on priority rules established by the one tissue processor.

15. One or more non-transitory computer-readable storage media having computer-executable instructions embodied thereon, wherein, when executed by at least one processor on a tissue screening (TS) computing device, the computer-executable instructions cause the at least one processor to:
store, in a database associated with the TS computing device, tissue screening data associated with a plurality of tissue processors, the tissue screening data including a set of tissue screening parameters for each donor tissue request received by an organ procurement organization (OPO) from the plurality of tissue processors;
receive, from a user computing device associated with the OPO, a search request to identify one or more tissue processors that satisfy at least one search condition;
compare the at least one search condition to each set of tissue screening parameters stored in the database to determine at least one tissue processor that matches the at least one search condition;
generate, based on the comparison, search results in response to the received request, the search results including the at least one tissue processor;
generate instructions for displaying a first graphical user interface on the user computing device, the first graphical user interface including the search results; and
transmit the generated instructions to the user computing device to cause the first graphical user interface to be displayed on the user computing device.

16. The one or more non-transitory computer-readable storage media of claim 15, wherein the at least one search condition includes a donor age provided in units of gestation weeks.

17. The one or more non-transitory computer-readable storage media of claim 15, wherein, when executed by the at least one processor on the TS computing device, the computer-executable instructions further cause the at least one processor to:
generate instructions for displaying a graphical interface widget on the user computing device, the graphical interface widget configured for inputting the at least one search condition in units of gestation weeks; and
transmit the generated instructions to the user computing device to cause the graphical interface widget to be displayed on the user computing device.

18. The one or more non-transitory computer-readable storage media of claim 15, wherein, when executed by the at least one processor on the TS computing device, the computer-executable instructions further cause the at least one processor to:
determine that a subset of the plurality of tissue processors match the at least one search condition; and
rank the tissue processors within the subset with respect to one another based on priority rules designated by the OPO.

19. The one or more non-transitory computer-readable storage media of claim 15, wherein, when executed by the at least one processor on the TS computing device, the computer-executable instructions further cause the at least one processor to:
determine that a subset of the plurality of donor tissue requests stored in the database match the at least one search condition; and
determine that the subset of donor tissue requests are associated with one tissue processor.

20. The one or more non-transitory computer-readable storage media of claim 19, wherein, when executed by the at least one processor on the TS computing device, the computer-executable instructions further cause the at least one processor to rank the donor tissue requests within the subset with respect to one another based on priority rules established by the one tissue processor.
